(19) United States
(12) Patent Application Publication
(10) Pub. No.: US 2009/0189086 A1
(43) Pub. Date: Jul. 30, 2009

(54) USE OF ARYL-OR ALKYLOXY-SUBSTITUTED PHTHALOCYANINES AS MARKING SUBSTANCES FOR LIQUIDS

(75) Inventors: Thomas Gessner, Heidelberg (DE); Sophia Ebert, Mannheim (DE); Rüdiger Sens, Ludwigshafen (DE); Martin Könemann, Mannheim (DE); Wolfgang Ahlers, Lambsheim (DE); Christos Vamvakaris, Mannheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, Ludwigshafen (DE)

(21) Appl. No.: 12/282,985

(22) PCT Filed: Mar. 7, 2007
(86) PCT No.: PCT/EP07/52122

§ 371 Date: Sep. 15, 2008

(30) Foreign Application Priority Data
Mar. 15, 2006 (EP) 06111161.3

(51) Int. Cl. G01N 21/64; C07D 487/22; C07C 255/51; C07D 209/44; C10L 1/232; G01N 21/85

(52) U.S. Cl. 250/458.1; 540/472; 558/419; 548/471; 8/521; 250/573

(57) ABSTRACT

Phthalocyanines of the formula (I)

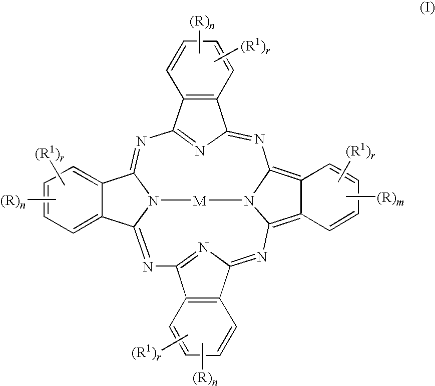

where the symbols and indices each have the definitions specified in the description are suitable as markers for liquids, especially mineral oils.

The invention relates to the use of specific aryl- or alkyloxy-substituted phthalocyanines as markers for liquids, especially mineral oils, to liquids, especially mineral oils, which comprise such a phthalocyanine as a marker, to a process for marking liquids and for detecting marked liquids, and to specific aryl- or alkyloxy-substituted phthalocyanines.

Among other compounds, WO 94/02570 A1 also proposes phthalocyanine derivatives as markers for liquids, especially mineral oils.

WO 98/52950 A1 describes phthalocyanines which comprise, as substituents, five- or six-membered, saturated, nitrogen-containing heterocyclic radicals which are bonded to the basic phthalocyanine skeleton via a ring nitrogen atom as markers for liquids, especially mineral oils.

Moreover, WO 2005/070935 describes phthalocyanines which bear substituents bonded via methylene groups on the basic phthalocyanine skeleton as markers for liquids, especially mineral oils.

In practice, it has been found that the known markers, especially in mineral oils, with the additives typically present therein, often do not have the desired long-term stability. As a result of the action of said additives, the characteristics (for example absorbance) of the markers change, so that there is still a great deal of room for improvement.

It is an object of the invention to provide phthalocyanines which feature not just good solubility but also good long-term stability in the liquids to be marked, especially mineral oils.

It has been found that certain aryl- or alkoxy-substituted phthalocyanines have both good solubility and good long-term stability, especially toward customary fuel additives.

The invention accordingly provides for the use of phthalocyanines of the formula (I) as markers for liquids

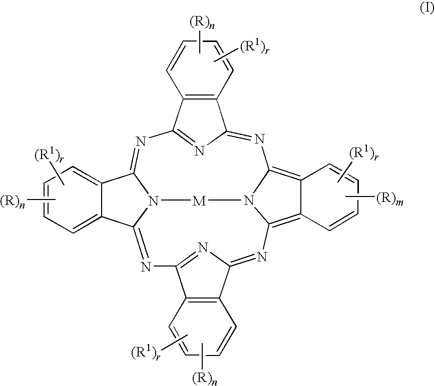

where the symbols and indices in the formula (I) have the following definitions:
M is twice hydrogen, twice lithium, magnesium, zinc, copper, nickel, VO, TiO, AlCl, AlOCOCH₃, AlOCOCF₃, SiCl₂ or Si(OH)₂;
m is 1, 2, 3 or 4;
n is the same or different and is 0, 1, 2, 3 or 4;
r is the same or different and is 0, 1, 2, 3 or 4;
m+r is 1, 2, 3 or 4;
n+r is 0, 1, 2, 3 or 4;
R is the same or different and is

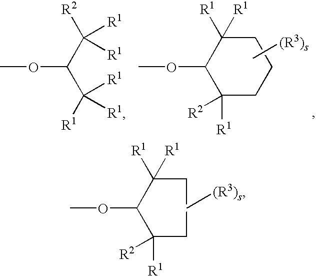

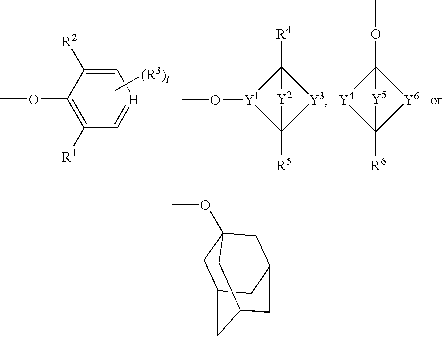

R¹ is the same or different and is H, halogen or R²;
R² is the same or different and is (C₁-C₁₈)-alkyl, (C₄-C₈)-cycloalkyl, (C₂-C₁₂)-alkenyl, (C₆-C₁₀)-aryl, (C₇-C₂₀)-aralkyl or (C₂-C₁₂)-alkynyl, where aryl radicals are unsubstituted or substituted by one or more halogen, cyano, nitro, hydroxyl, amino, C₁-C₂₀-alkyl which is optionally interrupted by from 1 to 4 oxygen atoms in ether function, C₁-C₂₀-alkoxy, C₁-C₂₀-alkylamino or C₁-C₂₀-dialkylamino;
R³ is the same or different and is R¹, or two R³ radicals or one R¹ radical and one R³ radical together form a further ring system;
R⁴, R⁵, R⁶ are the same or different and are each H, halogen, CH₃ or C₂H₅;
Y¹, Y², Y³, Y⁴, Y⁵, Y⁶ are the same or different and are each (C₁-C₄)-alkylene which is unsubstituted or substituted by one or more halogen atoms;
s is 0, 1, 2, 3, 4, 5, or 6; and
t is 0, 1, 2, 3.

C₁-C₁₈-Alkyl includes, for example, methyl, ethyl, propyl, isopropyl, butyl, isobutyl, sec-butyl, tert-butyl, pentyl, isopentyl, neopentyl, tert-pentyl, hexyl, 2-methylpentyl, heptyl, hept-3-yl, octyl, 2-ethylhexyl, isooctyl, nonyl, isononyl, decyl, isodecyl, undecyl, dodecyl, tridecyl, 3,5,5,7-tetramethylnonyl, isotridecyl (the above names isooctyl, isononyl, isodecyl and isotridecyl are trivial names and stem from the alcohols obtained by the oxo process—on this subject, cf. Ullmanns Encyklopädie der technischen Chemie [Encyclopedia of Industrial Chemistry], 4th edition, Volume 7, pages 215 to 217, and Volume 11, pages 435 and 436), tetradecyl, pentadecyl, hexadecyl, heptadecyl and octadecyl.

C₄-C₈-Cycloalkyl radicals include cyclobutyl, cyclopentyl, cyclohexyl, cycloheptyl and cyclooctyl.

C₆-C₁₀-Aryls include in particular phenyl and naphthyl. These are optionally substituted by one or more halogen atoms such as fluorine, chlorine or bromine, cyano, nitro, hydroxyl, amino, C₁-C₂₀-alkyl which is optionally interrupted by from 1 to 4 oxygen atoms in ether function, C₁-C₂₀-alkoxy, C₁-C₂₀-alkylamino or C₁-C₂₀-dialkylamino.

(C₇-C₂₀)-Aralkyls which, in the aryl radical, are optionally substituted by one or more halogen, cyano, nitro, hydroxyl, amino, C₁-C₂₀-alkyl which is optionally interrupted by from 1 to 4 oxygen atoms in ether function, C₁-C₂₀-alkoxy, C₁-C₂₀-alkylamino or C₁-C₂₀-dialkylamino are in particular benzyl, phenylethyl, 3-phenylpropyl and 4-phenylbutyl.

(C₂-C₁₂)-Alkenyl is understood in particular to mean propenyl, butenyl, pentenyl and hexenyl with their various positional isomers.

(C₂-C₁₂)-Alkynyl is understood in particular to mean propynyl, butynyl, pentynyl, hexynyl, heptynyl, octynyl, nonynyl, decynyl, undecynyl and dodecynyl with their various positional isomers.

Halogen is understood in particular to mean fluorine, chlorine, bromine and iodine.

The symbols and indices in the formula (I) preferably have the following definitions:
M is preferably twice hydrogen, twice lithium, magnesium, zinc, copper, nickel, VO, TiO, SiCl₂ or Si(OH)₂.
m is preferably 1 or 2.
n is preferably 0, 1 or 2.
r is preferably 0, 1 or 2.
R is preferably the same or different and is

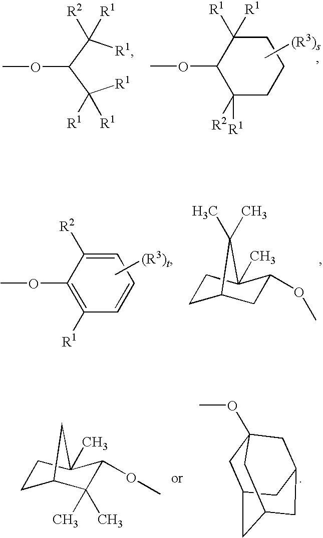

R¹ is preferably the same or different and is H or R².
R² is preferably the same or different and is (C₁-C₁₂)-alkyl, (C₅-C₇)-cycloalkyl, phenyl, (C₇-C₁₆)-aralkyl, where phenyl is unsubstituted or substituted by one or more halogen, (C₁-C₁₂)-alkyl or (C₁-C₁₂)-alkoxy.
R³ is preferably the same or different and is R¹.
s is preferably 0, 1 or 2.
t is preferably 0, 1 or 2.

Preference is given to compounds of the formula (I) in which all symbols and indices have the preferred definitions.

More preferably, the symbols and indices in the formula (I) have the following definitions:
M is more preferably twice hydrogen.
m is more preferably 1 or 2.
n is more preferably 1 or 2.
r is more preferably 0.
R is more preferably the same or different and is

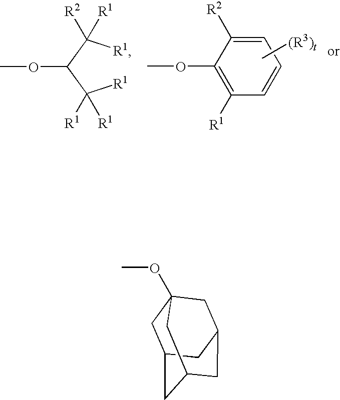

R¹ is more preferably the same or different and is H or R².
R² is more preferably the same or different and is (C₁-C₁₂)-alkyl, phenyl, (C₅-C₆)-cycloalkyl, where phenyl is unsubstituted or substituted by from one to three radicals from the group of F, Cl, (C₁-C₆)-alkyl and (C₁-C₆)-alkoxy.
R³ is more preferably the same or different and is R¹.
s is more preferably 0 or 1.
t is more preferably 0 or 1.

Particular preference is given to compounds of the formula (I) in which all symbols and indices have the particularly preferred definitions.

Most preferably, the symbols and indices in the formula (I) have the following definitions:
m is most preferably 1.
n is most preferably 1.
r is most preferably 0.
M is most preferably H.
R is most preferably

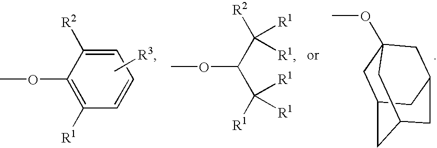

R¹ is most preferably the same or different and is H or R².
R² is most preferably (C₁-C₁₂)-alkyl or phenyl.
R³ is most preferably H or (C₁-C₁₂)-alkyl.

Most preference is given to the compounds of the formula (I) in which all symbols and indices have the most preferred definitions.

Preference is further given to the compounds of the formula (Ia)

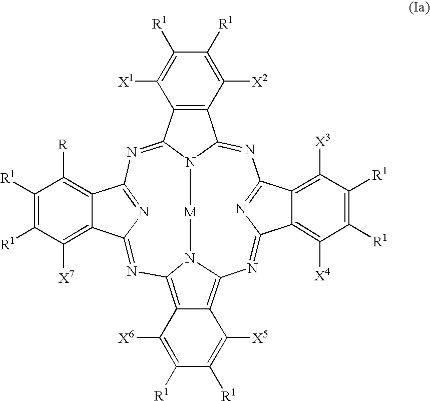

where the symbols have the following definitions:
X¹⁻⁷ are the same or different and are each R or R¹,
and
M, R and R¹ each have the definitions specified in the formula (I).

Particular preference is given to the compounds of the formula (Iaa)

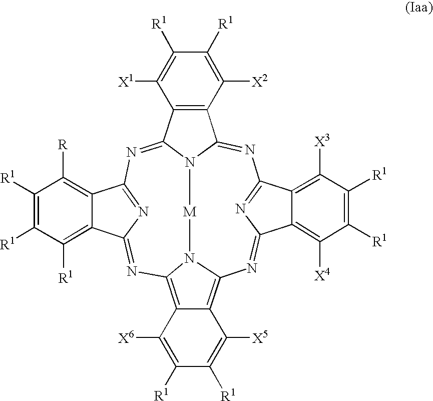

where in each case one of the two X¹ and X², X³ and X⁴, and X⁵ and X⁶ groups has the definition of R and the other has the definition of R¹, and
X¹-X⁶, R and R¹ are each as defined above.

Preferred compounds of the formula (Iaa) are those where all four R radicals have the same definition.

Preference is likewise given to compounds in which R¹ has the definition of H. Particularly preferred compounds of the formula (Iaa) are thus those in which all four R radicals have the same definition and R¹ has the definition of H.

Especially preferred are the isomeric compounds of the formulae (Iaaa), (Ibbb), (Iccc) and (Iddd), and also mixtures of these compounds, as can be formed, for example, in the synthesis of such compounds,

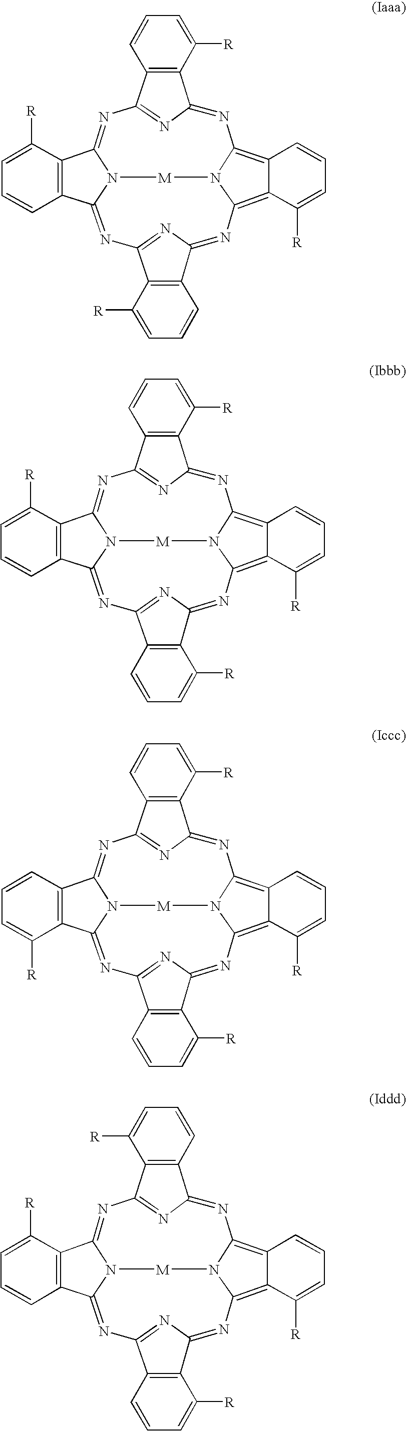

where M and R each have the definitions specified in the formula (I).

Also especially preferred are compounds of the formula (I) in which R has one of the following definitions

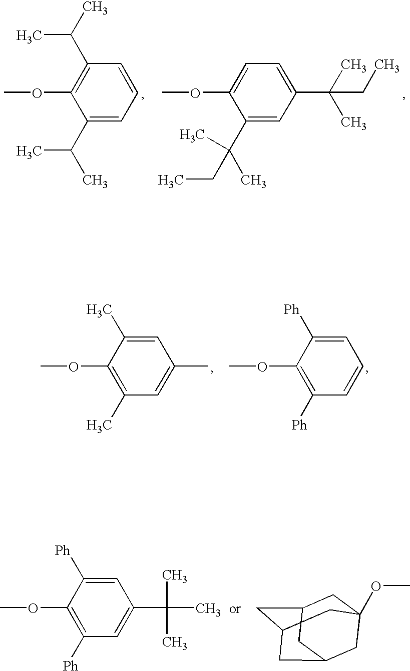

and also the compounds listed in the examples.

Some of the compounds of the formula (I) are known and some of them are novel.

The invention therefore also provides compounds of the formula (I) in which the symbols and indices are each defined as follows:

R is a group

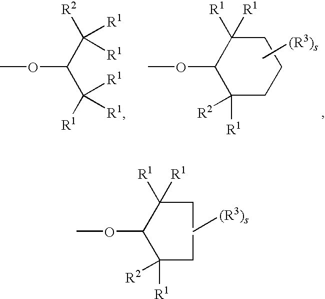

where the three groups above must each have at least 10 carbon atoms,

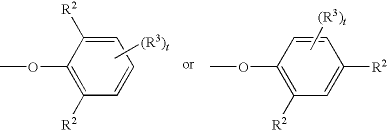

and the remaining symbols and indices each have the definitions specified in the formula (I).

The compounds of the formula (I) can be prepared by known methods familiar to the person skilled in the art, as described, for example, in F. H. Moser and A. L. Thomas in Phthalocyanine Compounds, ACS Monograph Series, Chapman & Hall, New York, 1963, F. H. Moser and A. L. Thomas in The Phthalocyanines, Manufacture and Applications, Vol. 2, CRC Press, Boca Raton, 1983, C. C. Leznoff in Phthalocyanines, Properties and Application (Eds.: C. C. Leznoff and A. B. P. Lever), Vol. 1, VCH, New York, Weinheim, Cambridge, 1989, M. Hanack, H. Heckmann and R. Polley in Houben-Weyl, Methods of Organic Chemistry (Ed.: E. Schaumann), 4th ed., Vol. E 9d, p. 727, Thieme, Stuttgart, New York, 1998, U.S. Pat. No. 3,509,146, EP-A 0 373 643, EP 0 658 604, EP-A 0 703 280, EP 0 848 040 and U.S. Pat. No. 6,348,250.

The invention also provides a process for preparing the abovementioned novel compounds of the formula (I), wherein a phthalonitrile of the formula (II)

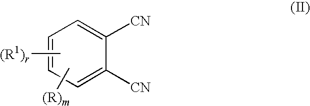

where the symbols and indices each have the definitions specified above is reacted with a reducing agent in the presence of a base in the melt.

Suitable reducing agents are, for example, hydroquinone, resorcinol, pyrocatechol and pyrogallol (1,2,3-trihydroxybenzene) or mixtures thereof, preference being given to hydroquinone.

Suitable bases are, for example, alkalimetal hydroxides, oxides and carbonates, preference being given to NaOH.

The molar ratio of phthalonitrile to reducing agent is generally from 0.1 to 10:1, preferably from 0.5 to 2:1.

In general, from 0.1 to 1 equivalent, preferably from 0.2 to 0.5 equivalent, of base is used.

The reaction is carried out in the melt, preferably at temperatures of from 140 to 250° C., more preferably from 150 to 200° C.

The reaction time is generally from 1 to 24 h.

The reaction is effected generally under atmospheric pressure, but may also be carried out at elevated or reduced pressure if appropriate.

The phthalonitriles of the formula (II) are likewise novel and form part of the subject-matter of the invention.

They can be prepared by known methods familiar to those skilled in the art, as described, for example, in EP-A 1 424 323 and EP-A 0 373 643.

The phthalonitriles (II) can be converted to the phthalocyanines of the formula (I) by the methods cited, if appropriate also via the iminoaminoisoindolines (III a/b) as isolated intermediates.

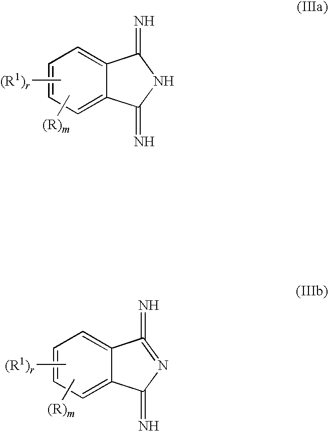

where the symbols and indices each have the definitions specified above. The compounds of the formula (III a/b) are novel and likewise form part of the subject-matter of the invention.

Suitable liquids which can be marked by means of the phthalocyanines of the formula (I) are in particular organic liquids, for example alcohols such as methanol, ethanol, propanol, isopropanol, butanol, isobutanol, sec-butanol, pentanol, isopentanol, neopentanol or hexanol, glycols such as 1,2-ethylene glycol, 1,2- or 1,3-propylene glycol, 1,2-, 2,3- or 1,4-butylene glycol, di- or triethylene glycol or di- or tripropylene glycol, ethers such as methyl tert-butyl ether, 1,2-ethylene glycol mono- or dimethyl ether, 1,2-ethylene glycol mono- or diethyl ether, 3-methoxypropanol, 3-iso-propoxypropanol, tetrahydrofuran or dioxane, ketones, such as acetone, methyl ethyl ketone or diacetone alcohol, esters such as methyl acetate, ethyl acetate, propyl acetate or butyl acetate, aliphatic or aromatic hydrocarbons such as pentane, hexane, heptane, octane, isooctane, petroleum ether, toluene, xylene, ethylbenzene, tetralin, decalin, dimethylnaphthalene, petroleum spirit, brake fluids or oils such as mineral oils which, in accordance with the invention, comprise petroleum, kerosene, diesel oil and heating oil, natural oils such as olive oil, soya oil or sunflower oil, or natural or synthetic motor, hydraulic or gearbox oils, for example motor vehicle oil or sewing machine oil.

The phthalocyanines of the formula (I) are used particularly advantageously for marking oils, especially mineral oils.

The invention additionally provides liquids, preferably oils, especially mineral oils, which comprise at least one phthalocyanine of the formula (I) as a marker.

The compounds of the formula (I) to be used as markers are added to the liquids in such amounts that reliable detection is ensured. Typically, the (weight-based) total content of markers in the marked liquid is from about 0.1 to 5000 ppb, preferably from 1 to 2000 ppb and more preferably from 1 to 1000 ppb.

To mark the liquids, the compounds are generally added in the form of solutions (stock solutions). Especially in the case of mineral oils, suitable solvents for preparing these stock solutions are preferably aromatic hydrocarbons such as toluene, xylene or higher-boiling aromatics mixtures.

In order to prevent too high a viscosity of such stock solutions (and hence poor dosability and handling), a total concentration of the markers of from 0.5 to 50% by weight, based on the total weight of these stock solutions, is generally selected.

The compounds of the formula (I) can, if appropriate, also be used in a mixture with other markers/dyes, as have been described, for example, at the outset. The total amount of the markers in the liquids is then typically within the range described above.

The invention also provides a process for marking liquids, preferably oils, especially mineral oils, wherein a compound of the formula (I) is added to the liquid.

The compounds of the formula (I) are detected in the liquids by common methods. Since these compounds generally have a high absorption capacity and/or exhibit fluorescence, one possibility in the given case is, for example, spectroscopic detection.

The compounds of the formula (I) generally have their absorption maximum in the range from 600 to 800 nm and/or fluoresce in the range from 600 to 900 nm and can thus be detected easily with suitable instruments.

The detection can be effected in a manner known per se, for example by measuring the absorption spectrum of the liquids to be analyzed.

It is also possible to excite the fluorescence of the compounds of the formula (I) present in the liquids, advantageously with a semiconductor laser or a semiconductor diode. It is particularly favorable to employ a semiconductor laser or a semiconductor diode having a wavelength in the spectral region from λₘₐₓ−100 nm to λₘₐₓ+20 nm. λₘₐₓ here means the wavelength of the absorption maximum of the marker. The wavelength of maximum emission is in the range from 620 to 900 nm.

The fluorescence light thus generated is advantageously detected with a semiconductor detector, especially with a silicon photodiode or a germanium photodiode.

The detection succeeds in a particularly advantageous manner when an interference filter and/or an edge filter (with a short-wavelength transmission edge in the range from λₘₐₓ to λₘₐₓ+80 nm) and/or a polarizer is also disposed upstream of the detector.

By means of the abovementioned compounds, it is possible in a very simple manner to detect marked liquids even when the compounds of the formula (I) are present only in a concentration of about 1 ppm (detection by absorption) or about 5 ppb (detection by fluorescence).

The present invention also provides a process for identifying liquids, preferably oils, in particular mineral oils, which comprise at least one compound of the formula (I) in an amount which is sufficient to induce detectable fluorescence on irradiation with a suitable wavelength, wherein
a) the liquid is irradiated with electromagnetic radiation of a wavelength of from 600 to 800 nm and
b) the excited fluorescence radiation is detected with a device for detecting radiation in the long-wavelength visible region or in the near infrared region.

The phthalocyanines of the formula (I) can also be used as a component in additive concentrates (also referred to hereinafter, following the relevant terminology, as “packages”) which, in addition to a carrier oil and a mixture of various fuel additives, generally also comprise dyes and, for invisible fiscal or manufacturer-specific marking, additionally markers. These packages enable the supply of various mineral oil distributors from one “pool” of unadditized mineral oil and the imparting of the company-specific additization, color and marking to the mineral oil with the aid of their individual packages not until, for example, during the transfer to appropriate storage vessels.

The components present in such packages are then in particular:
a) at least one phthalocyanine of the formula (I) or preferred embodiments thereof,
b) at least one carrier oil,
c) at least one additive selected from the group consisting of detergents, dispersants and valve seat wear-inhibiting additives,
d) and also, if appropriate, further additives and assistants.

The carrier oils used are typically viscous, high-boiling and in particular thermally stable liquids. They cover the hot metal surfaces, for example the intake valves, with a thin liquid film and thus prevent or delay the formation and deposition of decomposition products on the metal surfaces.

Carrier oils useful as component b) of the fuel and lubricant additive concentrates are, for example, mineral carrier oils (base oils), especially those of the Solvent Neutral (SN) 500 to 2000 viscosity class, synthetic carrier oils based on olefin polymers having M_{N}=from 400 to 1800, in particular based on polybutene or polyisobutene (hydrogenated or nonhydrogenated), on poly-alpha-olefins or poly(internal olefins) and also synthetic carrier oils based on alkoxylated long-chain alcohols or phenols. Adducts, to be used as carrier oils, of ethylene oxide, propylene oxide and/or butylene oxide to polybutyl alcohols or polyisobutene alcohols are described, for instance, in EP 277 345 A1; further polyalkene alcohol polyalkoxylates to be used are described in WO 00/50543 A1. Further carrier oils to be used also include polyalkene alcohol polyether amines, as detailed in WO 00/61708.

It is of course also possible to use mixtures of different carrier oils, as long as they are compatible with one another and with the remaining components of the packages.

Carburetors and intake systems of internal combustion engines, but also injection systems for fuel metering, are being contaminated to an increasing degree by impurities which are caused, for example, by dust particles from the air and uncombusted hydrocarbon residues from the combustion chamber.

To reduce or prevent these contaminations, additives (“detergents”) are added to the fuel to keep valves and carburetors or injection systems clean. Such detergents are generally used in combination with one or more carrier oils. The carrier oils exert an additional “wash function”, support and often promote the detergents in their action of cleaning and keeping clean, and can thus contribute to the reduction in the amount of detergents required.

It should also be mentioned here that many of the substances typically used as carrier oils display additional action as detergents and/or dispersants, which is why the proportion of the latter can be reduced in such a case. Such carrier oils having detergent/dispersant action are detailed, for instance, in the last-mentioned WO document.

It is also often impossible to clearly delimit the mode of action of detergents, dispersants and valve seat wear-inhibiting additives, which is why these compounds are listed in summary under component c). Customary detergents which find use in the packages are listed, for example, in WO 00/50543 A1 and WO 00/61708 A1 and include:

polyisobuteneamines which are obtainable according to EP-A 244 616 by hydroformylation of highly reactive polyisobutene and subsequent reductive amination with ammonia, monoamines or polyamines, such as dimethyleneaminopropylamine, ethylenediamine, diethylenetriamine, triethylenetetramine or tetraethylenepentamine,
poly(iso)buteneamines which are obtainable by chlorination of polybutenes or polyisobutenes having double bonds predominantly in the β- and γ-position and subsequent amination with ammonia, monoamines or the abovementioned polyamines,
poly(iso)buteneamines which are obtainable by oxidation of double bonds in poly(iso)butenes with air or ozone to give carbonyl or carboxyl compounds and subsequent amination under reducing (hydrogenating) conditions,
polyisobuteneamines which are obtainable according to DE-A 196 20 262 from polyisobutene epoxides by reaction with amines and subsequent dehydration and reduction of the amino alcohols,
polyisobuteneamines which optionally comprise hydroxyl groups and are obtainable according to WO-A 97/03946 by reaction of polyisobutenes having an average degree of polymerization P of from 5 to 100 with nitrogen oxides or mixtures of nitrogen oxides and oxygen and subsequent hydrogenation of these reaction products,
polyisobuteneamines which comprise hydroxyl groups and are obtainable according to EP-A 476 485 by reaction of polyisobutene epoxides with ammonia, monoamines or the abovementioned polyamines,
polyetheramines which are obtainable by reaction of C₂-C₃₀-alkanols, C₆-C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl or amino group and subsequent reductive amination with ammonia, monoamines or the abovementioned polyamines, and also
“polyisobutene Mannich bases” which are obtainable according to EP-A 831 141 by reaction of polyisobutene-substituted phenols with aldehydes and monoamines or the abovementioned polyamines.

Further detergents and/or valve seat wear-inhibiting additives to be used are listed, for example, in WO 00/47698 A1 and comprise compounds which have at least one hydrophobic hydrocarbon radical having a number-average molecular weight (M_{N}) of from 85 to 20 000 and at least one polar moiety, and which are selected from:
(i) mono- or polyamino groups having up to 6 nitrogen atoms, of which at least one nitrogen atom has basic properties;
(ii) nitro groups, optionally in combination with hydroxyl groups;
(iii) hydroxyl groups in combination with mono- or polyamino groups, in which at least one nitrogen atom has basic properties;
(iv) carboxyl groups or their alkali metal or alkaline earth metal salts;
(v) sulfonic acid groups or their alkali metal or alkaline earth metal salts;
(vi) polyoxy-C₂-C₄-alkylene moieties which are terminated by hydroxyl groups, mono- or polyamino groups, in which at least one nitrogen atom has basic properties, or by carbamate groups;
(vii) carboxylic ester groups;
(viii) moieties derived from succinic anhydride and having hydroxyl and/or amino and/or amido and/or imido groups; and
(ix) moieties obtained by Mannich reaction of phenolic hydroxyl groups with aldehydes and mono- or polyamines.

Additives comprising mono- or polyamino groups (i) are preferably polyalkenemono- or polyalkenepolyamines based on polypropene or on highly reactive (i.e. having predominantly terminal double bonds, usually in the β- and γ-positions) or conventional (i.e. having predominantly internal double bonds) polybutene or polyisobutene having M_{N}=from 300 to 5000. Such additives based on highly reactive polyisobutene, which can be prepared from the polyisobutene (which may comprise up to 20% by weight of n-butene units) by hydroformylation and reductive amination with ammonia, monoamines or polyamines, such as dimethylaminopropylamine, ethylenediamine, diethylenetriamine, triethylenetetramine or tetraethylenepentamine, are disclosed in particular in EP 244 616 A2. When polybutene or polyisobutene having predominantly internal double bonds (usually in the β- and γ-positions) are used as starting materials in the preparation of the additives, a possible preparative route is by chlorination and subsequent amination or by oxidation of the double bond with air or ozone to give the carbonyl or carboxyl compound and subsequent amination under reductive (hydrogenating) conditions. The amines used here for the amination may be the same as those used above for the reductive amination of the hydroformylated highly reactive polyisobutene. Corresponding additives based on polypropene are described in particular in WO 94/24231 A1.

Further preferred additives comprising monoamino groups (i) are the hydrogenation products of the reaction products of polyisobutenes having an average degree of polymerization P of from 5 to 100 with nitrogen oxides or mixtures of nitrogen oxides and oxygen, as described in particular in WO 97/03946 A1.

Further preferred additives comprising monoamino groups (i) are the compounds obtainable from polyisobutene epoxides by reaction with amines and subsequent dehydration and reduction of the amino alcohols, as described in particular in DE 196 20 262 A1.

Additives comprising nitro groups (ii), if appropriate in combination with hydroxyl groups, are preferably reaction products of polyisobutenes having an average degree of polymerization P of from 5 to 100 or from 10 to 100 with nitrogen oxides or mixtures of nitrogen oxides and oxygen, as described in particular in WO 96/03367 A1 and WO 96/03479 A1. These reaction products are generally mixtures of pure nitropolyisobutanes (e.g. α,β-dinitropolyisobutane) and mixed hydroxynitropolyisobutanes (e.g. α-nitro-β-hydroxypolyisobutane).

Additives comprising hydroxyl groups in combination with mono- or polyamino groups (iii) are in particular reaction products of polyisobutene epoxides obtainable from polyisobutene having preferably predominantly terminal double bonds and M_{N}=from 300 to 5000, with ammonia or mono- or polyamines, as described in particular in EP 476 485 A1.

Additives comprising carboxyl groups or their alkali metal or alkaline earth metal salts (iv) are preferably copolymers of C₂-C₄₀-olefins with maleic anhydride which have a total molar mass of from 500 to 20 000 and of whose carboxyl groups some or all have been converted to the alkali metal or alkaline earth metal salts and any remainder of the carboxyl groups has been reacted with alcohols or amines. Such additives are disclosed in particular by EP 307 815 A1. Such additives serve mainly to prevent valve seat wear and can, as described in WO 87/01126 A1, advantageously be used in combination with customary detergents such as poly(iso)buteneamines or polyetheramines.

Additives comprising sulfonic acid groups or their alkali metal or alkaline earth metal salts (v) are preferably alkali metal or alkaline earth metal salts of an alkyl sulfosuccinate, as described in particular in EP 639 632 A1. Such additives serve mainly to prevent valve seat wear and can be used advantageously in combination with customary detergents such as poly(iso)buteneamines or polyetheramines.

Additives comprising polyoxy-C₂-C₄-alkylene moieties (vi) are preferably polyethers or polyetheramines which are obtainable by reaction of C₂-C₆₀-alkanols, C₆-C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl group or amino group and, in the case of the polyetheramines, by subsequent reductive amination with ammonia, monoamines or polyamines. Such products are described in particular in EP 310 875 A1, EP 356 725 A1, EP 700 985 A1 and U.S. Pat. No. 4,877,416. In the case of polyethers, such products also have carrier oil properties. Typical examples of these are tridecanol butoxylates, isotridecanol butoxylates, isononylphenol butoxylates and polyisobutenol butoxylates and propoxylates and also the corresponding reaction products with ammonia.

Additives comprising carboxylic ester groups (vii) are preferably esters of mono-, di- or tricarboxylic acids with long-chain alkanols or polyols, in particular those having a minimum viscosity of 2 mm²/s at 100° C., as described in particular in DE 38 38 918 A1. The mono-, di- or tricarboxylic acids used may be aliphatic or aromatic acids, and particularly suitable ester alcohols or ester polyols are long-chain representatives having, for example, from 6 to 24 carbon atoms. Typical representatives of the esters are adipates, phthalates, isophthalates, terephthalates and trimellitates of isooctanol, of isononanol, of isodecanol and of isotridecanol. Additives which comprise moieties derived from succinic anhydride and have hydroxyl and/or amino and/or amido and/or imido groups (viii) are preferably corresponding derivatives of polyisobutenylsuccinic anhydride which are obtainable by reacting conventional or highly reactive polyisobutene having M_{N}=from 300 to 5000 with maleic anhydride by a thermal route or via the chlorinated polyisobutene. Particular interest attaches to derivatives with aliphatic polyamines such as ethylenediamine, diethylenetriamine, triethylenetetramine or tetraethylenepentamine. Such gasoline fuel additives are described in particular in U.S. Pat. No. 4,849,572.

Additives comprising moieties obtained by Mannich reaction of phenolic hydroxyl groups with aldehydes and mono- or polyamines (ix) are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or highly reactive polyisobutene having M_{N}=from 300 to 5000. Such “polyisobutene-Mannich bases” are described in particular in EP 831 141 A1.

For a more precise definition of the additives detailed individually, reference is explicitly made here to the disclosures of the abovementioned prior art documents.

Dispersants as component c) are, for example, imides, amides, esters and ammonium and alkali metal salts of polyisobutenesuccinic anhydrides. These compounds find use especially in lubricant oils, but sometimes also as detergents in fuel compositions.

Further additives and assistants which may, if appropriate, be present as component d) of the packages are

organic solvents, for example alcohols such as methanol, ethanol, propanol, isopropanol, butanol, isobutanol, sec-butanol, pentanol, isopentanol, neopentanol or hexanol, for example glycols such as 1,2-ethylene glycol, 1,2- or 1,3-propylene glycol, 1,2-, 2,3- or 1,4-butylene glycol, di- or triethylene glycol or di- or tripropylene glycol, for example ethers such as methyl tert-butyl ether, 1,2-ethylene glycol monomethyl ether or 1,2-ethylene glycol dimethyl ether, 1,2-ethylene glycol monoethyl ether or 1,2-ethylene glycol diethyl ether, 3-methoxypropanol, 3-isopropoxypropanol, tetra-hydrofuran or dioxane, for example ketones such as acetone, methyl ethyl ketone or diacetone alcohol, for example esters such as methyl acetate, ethyl acetate, propyl acetate or butyl acetate, for example lactams such as N-methylpyrrolidinone (NMP), for example aliphatic or aromatic hydrocarbons and also mixtures thereof such as pentane, hexane, heptane, octane, isooctane, petroleum ether, toluene, xylene, ethylbenzene, tetralin, decalin, dimethylnaphthalene or white spirit and, for example, mineral oil such as gasoline, kerosene, diesel oil or heating oil,
corrosion inhibitors, for example based on ammonium salts, having a tendency to form films, of organic carboxylic acids or of heterocyclic aromatics in the case of ferrous metal corrosion protection,
antioxidants or stabilizers, for example based on amines such as p-phenylene-diamine, dicyclohexylamine or derivatives thereof or on phenols such as 2,4-di-tert-butylphenol or 3,5-di-tert-butyl-4-hydroxyphenylpropionic acid,
demulsifiers,
antistats,
metallocenes such as ferrocene or methylcyclopentadienylmanganese tricarbonyl,
lubricity improvers (lubricity additives) such as certain fatty acids, alkenylsuccinic esters, bis(hydroxyalkyl) fatty amines, hydroxyacetamides or castor oil,
amines for reducing the pH of the fuel,
further markers other than phthalocyanines of the formula (I) and their preferred embodiments and
dyes.

The concentration of component a), i.e. of the at least one phthalocyanine of the formula (I) or preferred embodiments thereof, in the packages is typically selected in such a magnitude that, after addition of the package to the mineral oil, the desired concentration of marker(s) is present therein. Typical concentrations of the markers in the mineral oil are, for instance, in the range from 0.01 up to a few 10s of ppm by weight.

Component b), i.e. the at least one carrier oil, is present in the packages typically in a concentration of from 1 to 50% by weight, in particular from 5 to 30% by weight, and component c), i.e. the at least one detergent and/or the at least one dispersant, typically in a concentration of from 25 to 90% by weight, in particular from 30 to 80% by weight, based in each case on the total amount of components a) to c) and, if appropriate, d), the sum of the individual concentrations of components a) to c) and, if appropriate, d) adding up to 100% by weight.

When, as component d), corrosion inhibitors, antioxidants or stabilizers, demulsifiers, antistats, metallocenes, lubricity improvers and amines to reduce the pH of the fuel are present in the packages, the sum of their concentrations typically does not exceed 10% by weight, based on the total amount of the package (i.e. the total amount of components a) to c) and d)), the concentration of the corrosion inhibitors and demulsifiers being typically in the range of from in each case about 0.01 to 0.5% by weight of the total amount of the package.

When, as component d), additional organic solvents (i.e. not already introduced with the remaining components) are present in the packages, the sum of their concentrations typically does not exceed 20% by weight, based on the total amount of the package. These solvents generally stem from solutions of the markers and/or dyes, which are added to the packages instead of the pure markers and/or dyes with a view to more precise meterability.

When, as component d), further markers other than phthalocyanines of the formula (I) or preferred embodiments thereof are present in the packages, their concentration is in turn based on the content that they are to have after addition of the packages in mineral oil. That which was stated for component a) applies mutatis mutandis.

When, as component d), dyes are present in the inventive packages, their concentration is typically, for instance, between 0.1 to 5% by weight, based on the total amount of the package.

The invention will be illustrated in detail by the examples.

### EXAMPLE 1

### 1(4),8(1),15(18),22(25)-Tetrakis(2,6-diisopropylphenoxy)phthalocyanine

### a) 3-(2,6-Diisopropylphenoxy)phthalonitrile

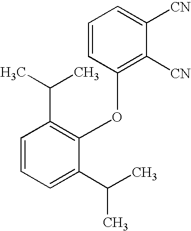

16.92 g (50.0 mmol) of cesium carbonate were added with stirring to a solution of 8.66 g (50.0 mmol) of 3-nitrophthalonitrile in 50 ml of N-methyl-2-pyrrolidinone. After the addition of 8.91 g (50.0 mmol) of 2,6-diisopropylphenol, the reaction mixture was heated to 40° C. and kept at this temperature for 24 hours. After cooling to room temperature, the reaction mixture was precipitated in 500 g of ice-water. The precipitate was filtered off with suction, washed with water and dried at 60° C. in a vacuum drying cabinet. The crude product (15.8 g) was dissolved in 200 ml of methanol, stirred at room temperature for 30 min and then precipitated with 800 ml of water. The precipitate was filtered off with suction, washed with 100 ml of water-methanol mixture (10:1) and dried at 60° C. in a vacuum drying cabinet. 11.17 g of solid were obtained. (A preparation method can also be found in M. Brewis et al., Chem. Eur. J. 1998, 4, 1633-1640.)

### b) 1(4),8(11),15(18),22(25)-Tetrakis(2,6-diisopropylphenoxy)phthalocyanine

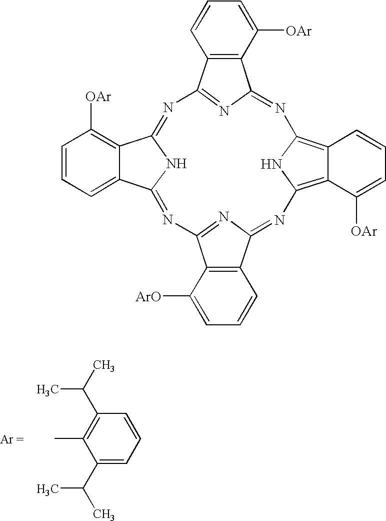

A mixture of 10.0 g (32.9 mmol) of 3-(2,6-diisopropylphenoxy)phthalonitrile, 3.63 g (33.0 mmol) of hydroquinone and 0.33 g (8.3 mmol) of sodium hydroxide granules were heated to 175° C. with stirring and kept at this temperature for 4 hours, the melt having solidified after 1 hour. After cooling to room temperature, the solid was comminuted and stirred with 200 ml of water and 10 ml of methanol. The solid was filtered off with suction, stirred in 200 ml of methanol, filtered off with suction and dried at 75° C. in a vacuum drying cabinet. The crude product was dissolved in toluene-heptane (2:1) and filtered through silica gel. The solution was concentrated to dryness and freed of solvent residues in a vacuum drying cabinet at 130° C. 3.01 g (30% of theory) of green microcrystals having a melting point of 229-231° C. (literature >300° C.) were obtained. (A preparation has already been described by M. Brewis et al., Chem. Eur. J. 1998, 4, 1633-1640.)

UV/Vis: λₘₐₓ(log ε)=726 (5.25), 692 (5.19), 660 (4.64), 626 (4.52), 354 (4.62), 318 nm (4.69) in toluene

λₘₐₓ (log ε)=726 (5.20), 694 (5.14), 662 (4.63), 628 (4.51), 352 (4.66), 316 nm (4.76) in methylene chloride

### EXAMPLE 2

### 1(4),8(11),15(18),22(25)-Tetrakis(2,4-di-tert-pentylphenoxy)phthalocyanine

### a) 3-(2,4-Di-tert-pentylphenoxy)phthalonitrile

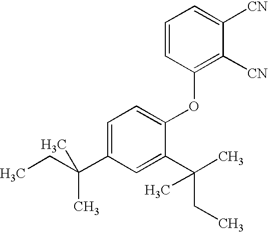

33.84 g (100 mmol) of cesium carbonate were added with stirring to a solution of 17.32 g (100 mmol) of 3-nitrophthalonitrile in 100 ml of N-methyl-2-pyrrolidinone. After the addition of 23.44 g (100 mmol) of 2,4-di-tert-pentylphenol, the reaction mixture was heated to 40° C. and kept at this temperature for 24 hours. After cooling to room temperature, the reaction mixture was precipitated in 1000 g of ice-water. The precipitate was filtered off with suction, washed with water and dried at 100° C. in a vacuum drying cabinet. The crude product (31.26 g) was recrystallized in 300 ml of methanol. The solid was filtered off with suction, washed with methanol and dried in a vacuum drying cabinet at 100° C. 23.75 g (66% of theory) of analytically pure microcrystals having a melting point of 143-144° C. (literature 133-135° C.) were obtained. (The preparation has also been described by G. Changsheng et al., Chinese J. Chem. Phys. 16 (2003) 293-298.)

### b) 1(4),8(11),15(18),22(25)-Tetrakis(2,4-di-tert-pentylphenoxy)phthalocyanine

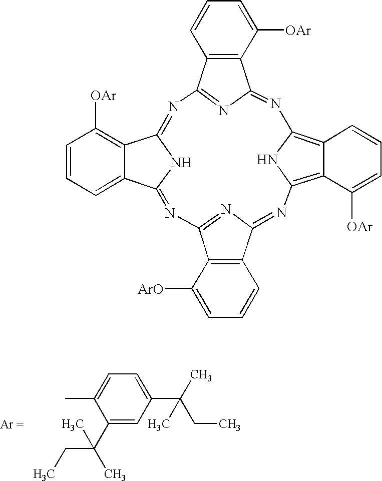

A mixture of 5.41 g (15.0 mmol) of 3-(2,4-di-tert-pentylphenoxy)phthalonitrile, 1.65 g (15.0 mmol) of hydroquinone and 0.15 g (3.6 mmol) of sodium hydroxide granules was heated to 175° C. with stirring and kept at this temperature for 4 hours, the melt having solidified after 1 hour. After cooling to room temperature, the solid was comminuted, dissolved in toluene-heptane (2:1) and filtered through silica gel. The solution was concentrated to dryness and freed of solvent residues in a vacuum drying cabinet at 130° C. 1.47 g (27% of theory) of green analytically pure microcrystals having a melting point of 230° C. (literature 230-232° C.) were obtained. (The preparation has also been described by G. Changsheng et al., Chinese J. Chem. Phys. 16 (2003) 293-298.)

UV/Vis: λₘₐₓ (log ε)=728 (5.26), 694 (5.21), 662 (4.66), 628 (4.55), 326 nm (4.74) in toluene

λₘₐₓ (log ε)=728 (5.21), 698 (5.16), 664 (4.64), 632 (4.54), 326 nm (4.79) in methylene chloride

### EXAMPLE 3

### 1(4),8(11),15(18),22(25)-Tetrakis(2,4,6-trimethylphenoxy)phthalocyanine

### a) 3-(2,4,6-Trimethylphenoxy)phthalonitrile

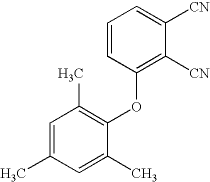

16.92 g (50.0 mmol) of cesium carbonate were added with stirring to a solution of 8.66 g (50.0 mmol) of 3-nitrophthalonitrile in 50 ml of N-methyl-2-pyrrolidinone. After the addition of 6.51 g (50.0 mmol) of 2,4,6-trimethylphenol, the reaction mixture was heated to 40° C. and kept at this temperature for 24 hours. After cooling to room temperature, the reaction mixture was admixed slowly with 100 ml of ice-water. The precipitate formed was filtered off with suction, washed with 100 ml of water and dried in a vacuum drying cabinet at 60° C. The crude product (12.03 g) was recrystallized in 200 ml of methanol. 6.12 g (45% of theory) of analytically pure colorless microcrystals having a melting point of 151-153° C. were obtained.

| | | | | | | | | | |
|---|---|---|---|---|---|---|---|---|---|
| C₁₇H₁₄N₂O | Calc. | C | 77.84 | H | 5.38 | N | 10.68 | O | 6.10 |
| M = 262.31 | Found. | C | 77.7 | H | 5.5 | N | 10.5 | O | 6.1 |

UV/Vis: λₘₐₓ(log ε)=316 (3.77), 308 (S) nm in acetonitrile

### b) 1(4),8(11),15(18),22(25)-Tetrakis(2,4,6-trimethylphenoxy)phthalocyanine

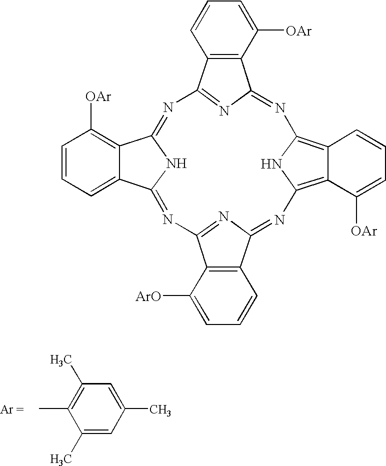

A mixture of 4.00 g (15.0 mmol) of 3-(2,4,6-trimethylphenoxy)phthalonitrile, 1.65 g (15.0 mmol) of hydroquinone and 0.16 g (4.0 mmol) of sodium hydroxide granules was heated to 175° C. with stirring and kept at this temperature for 4 hours, the melt having solidified after 1 hour. After cooling to room temperature, the solid (6.26 g) was comminuted, dissolved in toluene-heptane (2:1) and filtered through silica gel. The solution was concentrated to dryness and freed of solvent residues in a vacuum drying cabinet at 130° C. 1.07 g (27% of theory) of green microcrystals having a melting point of >370° C. were obtained.

| | | | | | | | |
|---|---|---|---|---|---|---|---|
| C₆₈H₅₈N₈O₄ | Calc. | C | 77.69 | H | 5.56 | N | 10.66 |
| M = 1051.27 | Found | C | 77.6 | H | 5.6 | N | 10.6 |

UV/Vis: λₘₐₓ (log ε)=724 (5.28), 690 (5.21), 658 (4.66), 624 (4.54), 354 (4.66), 320 nm (4.72) in toluene

### EXAMPLE 4

### 1(4),8(11),15(18),22(25)-Tetrakis(2,6-diphenylphenoxy)phthalocyanine

### a) 3-(2,6-Diphenylphenoxy)phthalonitrile (3-([1,1′;3′,1″]-terphenyl-2′-yloxy)-phthalonitrile)

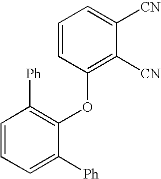

16.92 g (50.0 mmol) of cesium carbonate were added with stirring to a solution of 8.66 g (50.0 mmol) of 3-nitrophthalonitrile in 50 ml of N-methyl-2-pyrrolidinone. After the addition of 12.32 g (50.0 mmol) of 2,6-diphenylphenol the reaction mixture was heated to 40° C. and kept at this temperature for 24 hours. After cooling to room temperature, the reaction mixture was precipitated in 500 g of ice-water. The viscous precipitate was filtered off with suction and stirred up in 150 ml of ethanol. The finely crystalline precipitate was filtered off with suction, washed with ethanol and dried in a vacuum drying cabinet at 50° C. 1.43 g (7.7% of theory) of ochre-colored solid having a melting point of 129-130° C. (literature 128-129° C.) were obtained. (A preparation method can also be found in M. Brewis et al., Chem. Eur. J. 1998, 4, 1633-1640.)

### b) 1(4),8(11),15(18),22(25)-Tetrakis(2,6-diphenylphenoxy)phthalocyanine

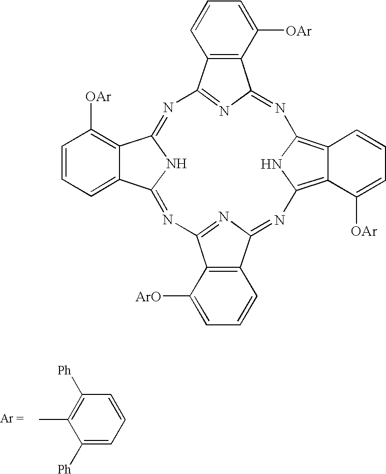

A mixture of 1.30 g (3.49 mmol) of 3-(2,6-diphenylphenoxy)phthalonitrile, 0.38 g (3.5 mmol) hydroquinone and 0.11 g (2.8 mmol) of sodium hydroxide granules was heated to 175° C. with stirring and kept at this temperature for 4 hours, the melt having solidified after 1 hour. After cooling to room temperature, the solid was comminuted. The crude product (1.75 g) was dissolved in toluene-heptane (2:1) and filtered through silica gel. The solution was concentrated to dryness and freed of solvent residues in a vacuum drying cabinet at 130° C. 0.49 g (38% of theory) of analytically pure green microcrystals having a melting point of 239-241° C. (literature >300° C.) was obtained. (A preparation has already been described by M. Brewis et al., Chem. Eur. J. 1998, 4, 1633-1640.)

UV/Vis: λₘₐₓ (log ε)=726 (5.25), 692 (5.18), 660 (4.62), 626 (4.50), 354 (4.60), 320 nm (4.66) in toluene

λₘₐₓ (log ε)=726 (5.21), 694 (5.15), 660 (4.62), 628 (4.50), 352 (4.64), 318 nm (4.72) in methylene chloride

### EXAMPLE 5

### 1(4),8(11),15(18),22(25)-Tetrakis(4-tert-butyl-2,6-diphenylphenoxy)-phthalocyanine

### a) 3-(4-tert-Butyl-2,6-diphenylphenoxy)phthalonitrile (3-(5′-tert-butyl-[1,1′;3′,1″]ter-phenyl-2′-yloxy)phthalonitrile)

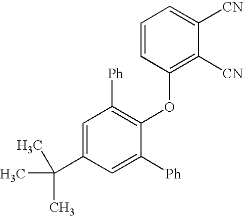

16.92 g (50.0 mmol) of cesium carbonate were added with stirring to a solution of 5.77 g (33.3 mmol) of 3-nitrophthalonitrile in 50 ml of N-methyl-2-pyrrolidinone. After the addition of 15.12 g (50.0 mmol) of 4-tert-butyl-2,6-diphenylphenol (prepared according to H. Yang and A. S. Hay, Synthesis 1992, 467-472), the reaction mixture was heated to 40° C. and stirred at this temperature for 6 hours. After cooling to room temperature, the reaction mixture was precipitated in 200 ml of water. The suspension was stirred over night and then filtered. The residue was suspended in 300 ml of ethanol and stirred at room temperature for 1 h. The solid was filtered off with suction, washed with ethanol and dried in a vacuum drying cabinet at 75° C. 10.66 g (75% of theory) of beige microcrystals were obtained. A sample recrystallized from ethanol (colorless) was analytically pure and melted at 189-191.5° C.

| | | | | | | | | | |
|---|---|---|---|---|---|---|---|---|---|
| C₃₀H₂₄N₂O | Calc. | C | 84.08 | H | 5.65 | N | 6.54 | O | 3.73 |
| M = 428.54 | Found | C | 83.8 | H | 5.7 | N | 6.3 | O | 3.9 |

UV/Vis: γₘₐₓ (log ε)=nm (3.71) in acetonitrile

### b) 1(4),8(11),15(18),22(25)-Tetrakis(4-tert-butyl-2,6-diphenylphenoxy)phthalocyanine

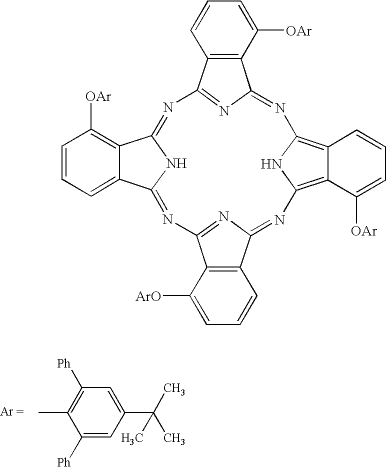

A mixture of 10.0 g (23.3 mmol) of 3-(4-tert-butyl-2,6-diphenylphenoxy)phthalonitrile, 2.57 g (23.3 mmol) of hydroquinone and 0.22 g (5.5 mmol) of sodium hydroxide granules was heated to 175° C. with stirring and kept at this temperature for 4 hours, the melt having solidified after 1 hour. After cooling to room temperature, the solid was comminuted. The crude product was stirred in 200 ml of water, filtered off with suction and dried at 100° C. in a vacuum drying cabinet. Subsequently, the solid was dissolved in toluene and purified chromatographically on silica gel. 1.86 g (19% of theory) of green microcrystals having a melting point of 243-245° C. were obtained.

| | | | | | | | | | |
|---|---|---|---|---|---|---|---|---|---|
| C₁₂₀H₉₈N₈O₄ | Calc. | C | 83.99 | H | 5.76 | N | 6.53 | O | 3.73 |
| M = 1716.17 | Found. | C | 83.9 | H | 6.2 | N | 6.7 | O | 3.6 |

UV/Vis: λₘₐₓ (log ε)=726 (5.29), 694 (5.23), 660 (4.66), 626 (4.54), 326 nm (4.70) in toluene

### EXAMPLE 6

### 2(3),9(10),16(17),23(24)-Tetrakis(2,6-diisopropylphenoxy)phthalocyanine

### a) 4-(2,6-Diisopropylphenoxy)phthalonitrile

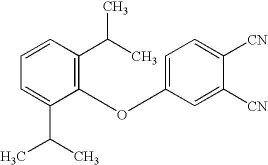

16.92 g (50.0 mmol) of cesium carbonate were added with stirring to a solution of 8.65 g (50.0 mmol) of 4-nitrophthalonitrile in 50 ml of N-methyl-2-pyrrolidinone. After the addition of 8.91 g (50.0 mmol) of 2,6-diisopropylphenol, the reaction mixture was heated to 40° C. and kept at this temperature for 24 hours. After cooling to room temperature, the reaction mixture was precipitated in 500 g of ice-water. The precipitate was filtered off with suction, washed with water and dried in a vacuum drying cabinet at 100° C. The crude product (6.53 g) was dissolved in 100 ml of hot ethanol. The hot solution was filtered and, after cooling to room temperature, precipitated with 300 ml of ice-water. The precipitate was filtered off with suction, washed with water and dried in a vacuum drying cabinet at 80° C. 3.81 g (25% of theory) of solid having a melting point of 114-116° C. (literature 115-116° C.) were obtained. (A preparation method can also be found in M. Brewis et al., Chem. Eur. J. 1998, 4, 1633-1640.)

### b) 2(3),9(10),16(17),23(24)-Tetrakis(2,6-diisopropylphenoxy)phthalocyanine

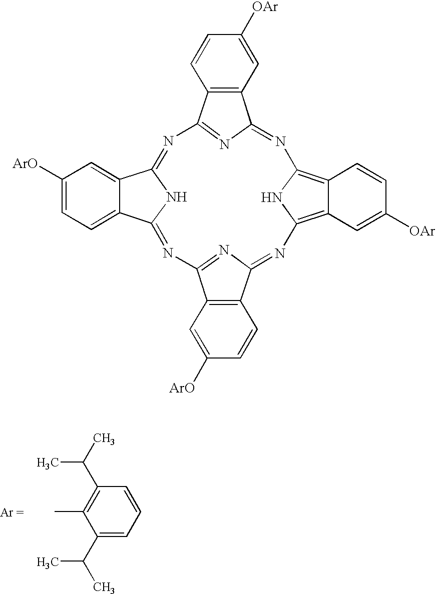

A mixture of 3.04 g (10.0 mmol) of 4-(2,6-diisopropylphenoxy)phthalonitrile, 1.10 g (10.0 mmol) of hydroquinone and 0.11 g (2.8 mmol) of sodium hydroxide granules was heated to 175° C. with stirring and kept at this temperature for 4 hours, the melt having been solidified after 1 hour. After cooling to room temperature, the solid was comminuted, dissolved in toluene-heptane (2:1) and filtered through silica gel. The solution was concentrated to dryness and freed of solvent residues in a vacuum drying cabinet at 130° C. 0.13 g (4% of theory) of green microcrystals having a melting point of 196-198° C. (literature >300° C.) was obtained. (A preparation has already been described by M. Brewis et al., Chem. Eur. J. 1998, 4, 1633-1640.)

UV/Vis: λₘₐₓ (log ε)=705 (5.23), 668 (5.14), 640 (4.67), 606 (4.49), 350 (4.82), 284 (4.63) nm in toluene

### EXAMPLE 7

### 1(4),8(11),15(18),22(25)-Tetra(1-adamantanoxy)phthalocyanine

### a) 3-(1-Adamantanoxy)phthalonitrile

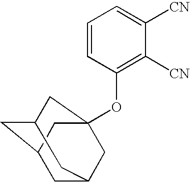

8.66 g (50.0 mmol) of 3-nitrophthalonitrile were dissolved in 50 ml of anhydrous N-methyl-2-pyrrolidinone (NMP) under nitrogen. A solution of sodium adamantoxide in anhydrous NMP, which had been prepared from 7.61 g (50.0 mmol) of 1-adamantanol and 2.20 g (55.0 mmol) of sodium hydride, was added dropwise to the solution cooled to 0-5° C. After stirring at 0-5° C. for two hours, the reaction solution was allowed to warm to room temperature and stirred for a further 18 hours. Subsequently, 150 ml of water were added dropwise, in the course of which a precipitate formed. This was filtered off with suction, washed with water and dried in a vacuum drying cabinet at 50° C. The crude product (8.08 g) was recrystallized from 80 ml of ethanol. 5.55 g of solid were obtained.

### b) 1(4),8(11),15(18),22(25)-Tetra(1-adamantanoxy)phthalocyanine

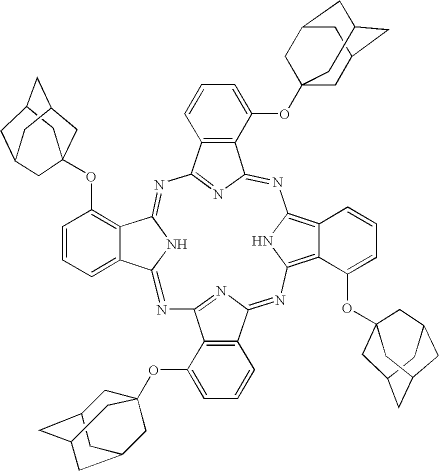

A mixture of 4.18 g (15.0 mmol) of 3-(1-adamantanoxy)phthalonitrile, 1.65 g (15.0 mmol) of hydroquinone and 0.15 g (3.6 mmol) of sodium hydroxide granules was heated to 175° C. with stirring and kept at this temperature for 4 hours, the melt having solidified after 1 hour. After cooling to room temperature, the solid was comminuted, dissolved in toluene-ethyl acetate (15:2) and filtered through silica gel. The solution was concentrated to dryness and freed of solvent residues in a vacuum drying cabinet at 130° C. 0.68 g (16% of theory) of green microcrystals having a melting point of 124-125° C. was obtained.

UV/Vis: λₘₐₓ(log ε)=718 (5.08), 684 (5.01), 652 (4.49), 620 (4.34), 356 (4.58), 310 nm (4.50) in toluene

### EXAMPLE 8 (Comparative Example)

### 1(4),8(11),15(18),22(25)-tetra(4-nonylphenoxy)-phthalocyanine

### a) 3-(4-Nonylphenoxy)phthalonitrile

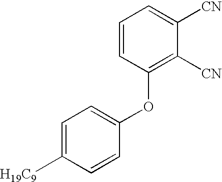

124.4 g (900 mmol) of potassium carbonate were added with stirring to a solution of 155.13 g (600 mmol) of 3-nitrophthalonitrile in 500 ml of dimethylformamide. After the addition of 132.1 g (600 mmol) of 4-nonylphenol, the reaction mixture was warmed to 35° C. and kept at this temperature for 6 hours. Subsequently, the reaction mixture was stirred at room temperature over night and then precipitated in 6 l of water. The precipitate formed was filtered off with suction, washed with 6 l of water and dried at 40° C. in a vacuum drying cabinet. The crude product (194.3 g) was recrystallized in 1 l of n-hexane and then in 200 ml of methanol in the presence of activated carbon. 41.0 g (20% of theory) of colorless microcrystals having a melting point of 74-81° C. were obtained.

### b) 1(4),8(11),15(18),22(25)-Tetra(4-nonylphenoxy)phthalocyanine

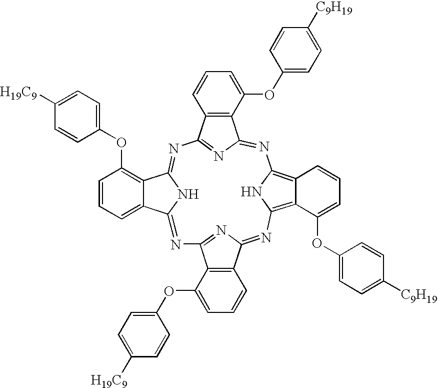

A mixture of 34.6 g (100 mmol) of 3-(4-nonylphenoxy)phthalonitrile, 1.11 g (100 mmol) of hydroquinone and 1.00 g (25.0 mmol) of sodium hydroxide granules was heated to 175° C. with stirring and kept at this temperature for 4 hours. After cooling to room temperature, the melt solidified. The solid (36.0 g) was triturated finely, slurried with 200 ml of water and 10 ml of ethanol, filtered off with suction, washed with 1 l of water and dried in a vacuum drying cabinet at 60° C. The crude product (34.6 g) was dissolved in 210 ml of toluene. The solution was filtered and added dropwise to 700 ml of methanol. After stirring for one hour, the solid was filtered off with suction, washed with 700 ml of methanol then with water, and dried in a vacuum drying cabinet at 60° C. (19.4 g). The solid was recrystallized in 194 ml of butylglycol. The solid was filtered off with suction, washed with 40 ml of butylglycol then with 390 ml of methanol, suction-dried and dried at 60° C. in a vacuum drying cabinet. 15.4 g (44% of theory) of analytically pure green microcrystals having a melting point of 168.5-170° C. were obtained.

| | | | | | | | |
|---|---|---|---|---|---|---|---|
| C₉₂H₁₀₆N₈O₄ | Calc. | C | 79.62 | H | 7.70 | N | 8.07 |
| M = 1387.90 | Found | C | 79.5 | H | 7.6 | N | 8.2 |

UV/Vis: λₘₐₓ (log ε)=718 (5.20), 684 (5.14), 654 (4.66), 620 (4.52), 330 nm in toluene

### EXAMPLE 9

### Storage Stability Testing in the Presence of Mineral Oil Additives

Approx. 20 mg of the particular substance were dissolved in 25 ml of Shellsol Naphtha heavy. Any insoluble constituents were removed by filtration (fluted filter). The concentration of the dissolved substance was selected such that the absorbances to be measured for the longest-wavelength absorption bands were, as far as possible, between 0.8 and 1.5. 5 ml of the filtrate were made up to 10 ml with a commercial additive based on polyisobutenamine (PIBA), mixed and stored at 40° C. in an ampule with airtight seal. After the storage times listed in the table below, samples were taken from the ampules and analyzed in 1 mm cuvettes. In order to obtain better comparability of the different samples, absorbances normalized to 1 (absorbence equal to 1 at the start of the storage time) are reported in the table.

| | | Storage | | |
|---|---|---|---|---|
| | | time | Normalized | UV/Vis |
| Substance | Additive | [h] | absorbance | λₘₐₓ [nm] |
| Example 1 | Kerocom ® PIBA 03 | 0 | 1 | 726 |
| | | 627 | 0.81 | 726 |
| Example 4 | Kerocom ® PIBA 03 | 0 | 1 | 728 |
| | | 646 | 1.00 | 728 |
| Example 5 | Kerocom ® PIBA 03 | 0 | 1 | 726 |
| | | 815 | 0.94 | 726 |
| Example 6 | Kerocom ® PIBA 03 | 0 | 1 | 706 |
| | | 815 | 0.96 | 706 |
| Example 8 | Kerocom ® PIBA 03 | 0 | 1 | 718 |
| (comparative | | 142 | 0.28 | 718 |
| example) | | | | |

1-13. (canceled)

14. A method for marking a liquid, comprising the step of adding to the liquid as a marker a phthalocyanine of the formula (I) 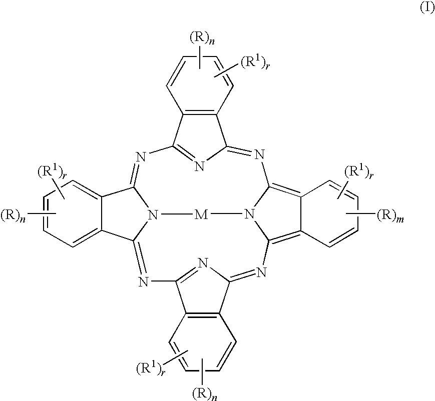
where the symbols and indices in the formula (I) have the following definitions:
M is twice hydrogen, twice lithium, magnesium, zinc, copper, nickel, VO, TiO, AlCl, AlOCOCH₃, AlOCOCF₃, SiCl₂ or Si(OH)₂;
m is 1, 2, 3 or 4;
n is the same or different and is 0, 1, 2, 3 or 4;
r is the same or different and is 0, 1, 2, 3 or 4;
m+r is 1, 2, 3 or 4;
n+r is 0, 1, 2, 3 or 4;
R is the same or different and is 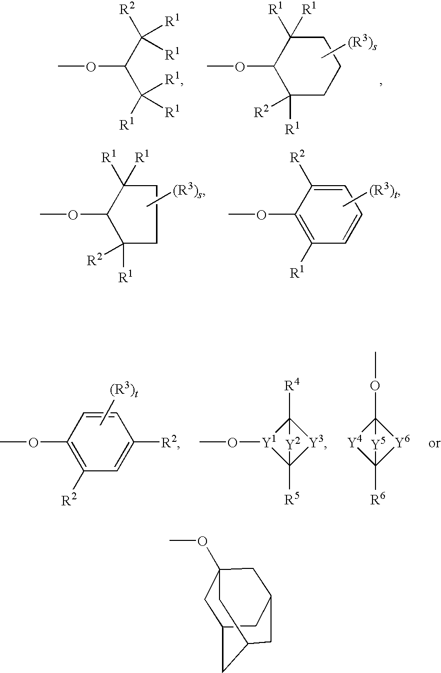
R¹ is the same or different and is H, halogen or R²;
R² is the same or different and is (C₁-C₁₈)-alkyl, (C₄-C₈)-cycloalkyl, (C₂-C₁₂)-alkenyl, (C₈-C₁₀)-aryl, (C₇-C₂₀)-aralkyl or (C₂-C₁₂)-alkynyl, where aryl radicals are unsubstituted or substituted by one or more halogen, cyano, nitro, hydroxyl, amino, C₁-C₂₀-alkyl which is optionally interrupted by from 1 to 4 oxygen atoms in ether function, C₁-C₂₀-alkoxy, C₁-C₂₀-alkylamino or C₁-C₂₀-dialkylamino;
R³ is the same or different and is R¹, or two R³ radicals or one R¹ radical and one R³ radical together form a further ring system;
R⁴, R⁵, R⁶ are the same or different and are each H, halogen, CH₃ or C₂H₅;
Y¹, Y², Y³, Y⁴, Y⁵, Y⁶ are the same or different and are each (C₁-C₄)-alkylene which is unsubstituted or substituted by one or more halogen atoms;
s is 0, 1, 2, 3, 4, 5, or 6; and
t is 0, 1, 2, 3.

15. The method according to claim 14, wherein the symbols and indices in the formula (I) have the following definition:
M is twice hydrogen, twice lithium, magnesium, zinc, copper, nickel, VO or TiO;
m is 1 or 2;
n is 0, 1 or 2;
r is 0, 1 or 2;
R is the same or different and is 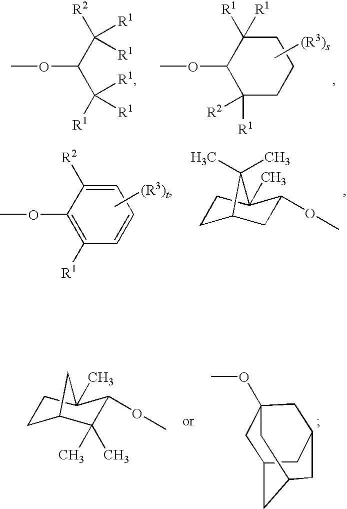
R¹ is the same or different and is H or R²;
R² is the same or different and is (C₁-C₁₂)-alkyl, (C₅-C₇)-cycloalkyl, phenyl, (C₇-C₁₆)-aralkyl, where phenyl is unsubstituted or substituted by one or more halogen, (C₁-C₁₂)-alkyl or (C₁-C₁₂)-alkoxy;
R³ is the same or different and is R¹;
s is 0, 1 or 2; and
t is 0, 1 or 2.

16. The method according to claim 14, wherein the symbols and indices in the formula (I) have the following definitions:
M is twice hydrogen,
m is 1 or 2;
n is 1 or 2;
r is 0;
R is the same or different and is 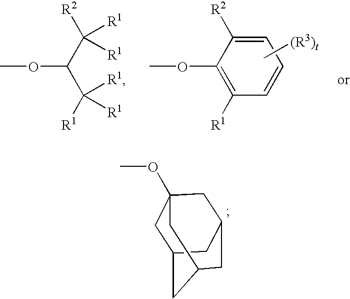
R¹ is the same or different and is H or R²;
R² is the same or different and is (C₁-C₁₂)-alkyl, phenyl, (C₅-C₆)-cycloalkyl, where phenyl is unsubstituted or substituted by from one to three radicals from the group of F, Cl, (C₁-C₆)-alkyl and (C₁-C₆)-alkoxy;
R³ is the same or different and is R¹;
s is 0 or 1; and
t is 0 or 1.

17. The method according to claim 14, wherein the symbols and indices in the formula (I) have the following definitions:
m is 1;
n is 1;
r is 0;
M is H;
R is 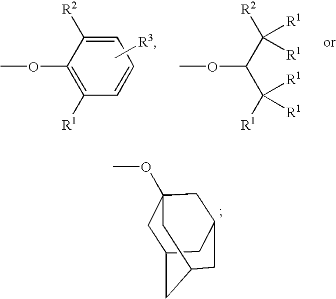
R¹ is the same or different and is H or R²;
R² is (C₁-C₁₂)-alkyl or phenyl;
R³ is H or (C₁-C₁₂)-alkyl.

18. The method according to claim 14, wherein the compounds of the formula (I) used are compounds of the formula (Ia) 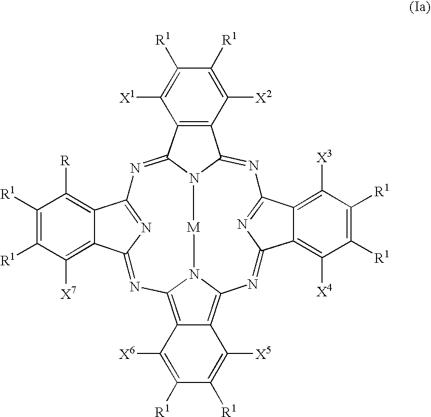
where the symbols have the following definitions:
X¹⁻⁷ are the same or different and are each R or R¹,
and
M, R and R¹ each have the definitions specified in the formula (I) in claim 14.

19. The method according to claim 14, wherein the liquid is a mineral oil.

20. A liquid comprising one or more phthalocyanines of the formula (I) according to claim 14 as a marker.

21. The liquid according to claim 20, wherein the liquid is a mineral oil.

22. A process for identifying liquids which comprise at least one compound of the formula (I) according to claim 14 in an amount which is sufficient to induce detectable fluorescence on irradiation with a suitable wavelength, wherein
a) the liquid is irradiated with electromagnetic radiation of a wavelength of from 600 to 800 nm and
b) the excited fluorescence radiation is detected with a device for detecting radiation in the long-wavelength visible region or in the near infrared region.

23. A phthalocyanine of the formula (I) 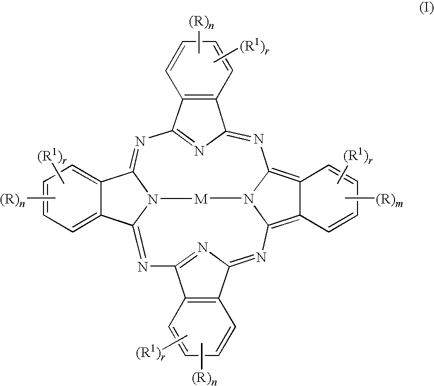
where the symbols and indices in the formula (I) have the following definitions:
M is twice hydrogen, twice lithium, magnesium, zinc, copper, nickel, VO, TiO, AlCl, AlOCOCH₃, AlOCOCF₃, SiCl₂ or Si(OH)₂;
m is 1, 2, 3 or 4;
n is the same or different and is 0, 1, 2, 3 or 4;
r is the same or different and is 0, 1, 2, 3 or 4;
m+r is 1, 2, 3 or 4;
n+r is 0, 1, 2, 3 or 4;
R is a group 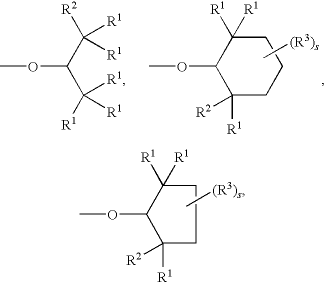
where the three groups above must each have at least 10 carbon atoms, 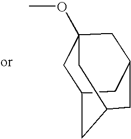
and R¹, R², R³ and s have the meaning given in formula (I) in claim 14.

24. A process for preparing phthalocyanines of the formula (I) according to claim 23, wherein a phthalonitrile of the formula (II), 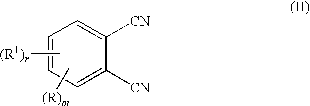
where the symbols and indices have the same definitions in the formula (I) according to claim 23 is reacted with a reducing agent in the presence of a base in the melt.

25. A phthalonitrile of the formula (II) according to claim 24.

26. An iminoaminoisoindoline of the formula (IIIa) or (IIIb) 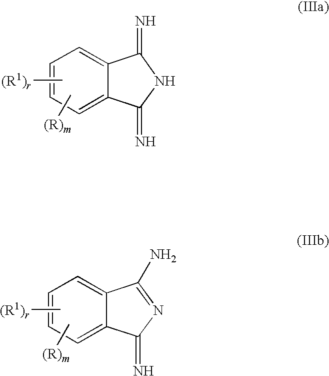
where the symbols and indices each have the definitions specified in the formula (II) in claim 23.
